(19) United States
(12) Patent Application Publication
(10) Pub. No.: US 2004/0254146 A1
(43) Pub. Date: Dec. 16, 2004

(54) Carboxylate salts of galantamine and their pharmaceutical use

(75) Inventors: Steven C. Quay, Edmonds, WA (US); Henry R. Costantino, Woodinville, WA (US); Michael E. Houston JR., Sammamish, WA (US); Alexis Kays Leonard, Shoreline, WA (US)

Correspondence Address: Nastech Pharmaceutical Company Inc., 3450 Monte Villa Parkway, Bothell, WA, 98021-8906, US

(73) Assignee: Nastech Pharmaceutical Company Inc.

(21) Appl. No.: 10/831,031

(22) Filed: Apr. 23, 2004

Related U.S. Application Data
(63) Continuation-in-part of application No. 10/439,108, filed on May 15, 2003.
(60) Provisional application No. 60/382,122, filed on May 21, 2002.

(51) Int. Cl. A61K 31/7008; C07H 5/06

(52) U.S. Cl. 514/62; 536/18.7

(57) ABSTRACT

Disclosed are novel carboxylate salts of galantamine including galantamine gluconate, galantamine lactate, galantamine citrate and galantamine glucarate. These salts of galantamine have more than a 5 fold increase in solubility compared to galantamine hydrobromide. These galantamine salts can be administered to an individual to inhibit acetylcholinesterase in the treatment of such diseases as Alzheimer's disease, atony of the smooth muscle of the intestinal tract and urinary bladder, glaucoma, myasthenia gravis, and termination of the effects of competitive neuromuscular blocking drugs.

This is a continuation-in-part of and claims priority under Title 35, U.S.Code, §120 to co-pending U.S. patent application Ser. No. 10/439,108 filed on May 15, 2003, which claims the benefit under 35 U.S.C §119(e) of U.S. Provisional Application No. 60/382,122 filed on May 21, 2002, the entire contents of both are incorporated herein by reference.

### BACKGROUND OF THE INVENTION

Galantamine, an acetylcholinesterase inhibitor, is an important drug for the prevention and treatment of diseases and disorders of the central nervous system. These diseases include, inter alia, neurological conditions associated with memory loss, cognitive impairment and dementia in mammals, including Alzheimer's Disease, Parkinson's-type dementia, certain forms of schizophrenia, forms of delirium, and dementia. Pathological changes in Alzheimer's disease involve, for example, degeneration of cholinergic neurons in the subcortical regions and of neuronal pathways that project from the basal forebrain. These pathways are thought to be intricately involved in memory, attention, learning, and other cognitive processes.

It is believed that acetylcholinesterase inhibitors exert their therapeutic effect in the central nervous system by enhancing cholinergic function, i.e., by increasing the concentration of acetylcholine through reversible inhibition of its enzymatic hydrolysis by the cholinesterases. This pharmacotherapeutic approach also has some value in treatment of nicotine withdrawal and sleep apnea, as well as the dementia and delirium states described above.

Currently, galantamine is delivered orally as the hydrobromide salt in tablet form or oral solution. However, as an orally administered drug, galantamine reaches a maximum inhibition of acetylcholinesterase one hour after administration. It is possible that an intranasal formulation could result in a maximum inhibition of acetylcholinesterase in a shorter amount of time than the orally administered galantamine. However, to intranasally deliver therapeutically relevant doses of galantamine, the concentration of drug would have to be in excess of 40 mg/mL, for example, preferably 80 mg/mL. This is dictated by the volume limitation for nasal spray dosing (˜100 μL per nostril per spray). However, the solubility of the currently available form, namely galantamine hydrobromide, does not achieve this goal. Thus, there is a need to produce formulations of galantamine that have increased solubility.

### BRIEF DESCRIPTION OF THE DRAWING

FIG. 1 shows the ultraviolet light (UV) absorbance of the galantamine fractions of Example 6.

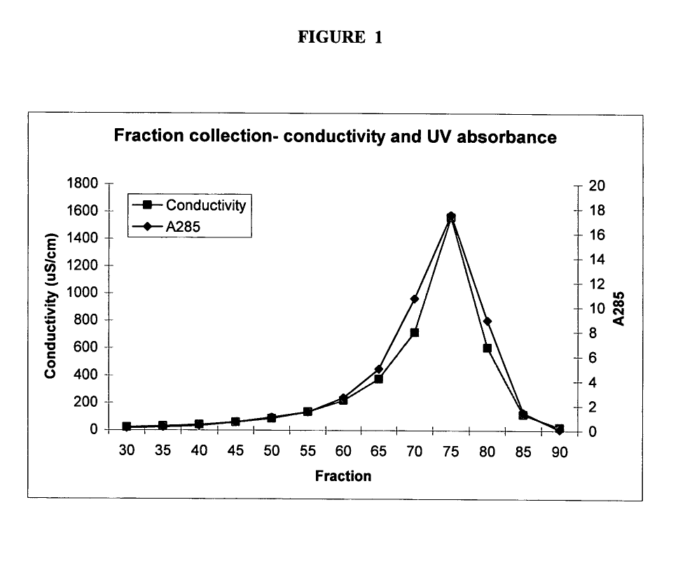

### DESCRIPTION OF THE INVENTION

The present invention fills this need by providing for novel galantamine carboxylate salts such as galantamine gluconate, galantamine lactate, galantamine glucarate and galantamine citrate. It has been unexpectedly discovered that the novel galantamine carboxylate salts of the present invention are substantially more soluble than galantamine hydrobromide.

In another embodiment of the present invention, carboxylate salts of galantamine of the present invention are produced by replacing the bromide of galantamine hydrobromide with a carboxylate anion which (1) provides higher solubility compared to bromide and (2) is a weaker anion than bromide (as illustrated by behavior upon ion exchange, described below). Examples of appropriate counter anions are carboxylates of the form:

R—(COO⁻)ₓ

where x≧1 and R is an alkyl group. In one embodiment, R contains one or more hydroxyl groups on the carbon backbone. Examples of such preferred embodiments include, but are not limited to, gluconate, lactate, glucarate, benzoate, acetate, salicylate, tartrate, mesylate, tosylate, maleate, fumarate, stearate and citrate.

Thus, the present invention is further encompassed by a method for producing a galantamine carboxylate salt in which a solution of a carboxylate salt formed producing carboxylate anions in solution. This solution containing the carboxylate anions is applied to an anion exchange resin under conditions wherein the carboxylate anions bind to the anion exchange resin. Galantamine hydrobromide is dissolved in an appropriate solvent such as water under conditions where bromide ions are formed in solution. The galantamine hydrobromide solution is then added to the anion exchange resin under conditions wherein the carboxylate anions are displaced and the bromide anions bind to the anion exchange resin resulting in the formation of a galantamine carboxylate salt or complex. Common types of anion exchange resins are diethylaminoethyl (DEAE-) and quaternary amino ethyl- (TEAE-, QAE) substituents attached directly hydroxyl groups on the matrix of the resin. Suitable ion exchange processes include, but are not limited to, batch processes using a resin slurry, and also a process using a resin packed in a column.

The current invention encompasses salt forms of galantamine with increased solubility compared to galantamine hydrobromide and methods for their generation. Said generation can be accomplished, for example, by salt exchange on an anion exchange resin, generally used for purification of proteins and peptides. Taking advantage of its strong-anion binding capability, a quaternary ammonium anion exchange resin is first saturated with R—(COO⁻)ₓ. After this weak anion is bound to the resin, galantamine hydrobromide is loaded on the resin. The bromide, being a stronger anion, displaces R—(COO⁻)ₓ on the resin and the galantamine elutes with a new, and more soluble, salt form. Elemental analysis confirmed 900 fold depletion of bromide in the eluted fractions from the resin. Water can then be removed to concentrate the new galantamine salt.

The present invention facilitates the development of nasal formulations by removing a previously existing barrier of concentration limitations. With the new salt forms, solubility can be increased at least ten fold over the concentration of galantamine hydrobromide. The maximum concentration of galantamine hydrobromide in water as about 35 mg/mL (121 mM). The generally reported solubility of galantamine hydrobromide in water is 50 mM. Surprisingly the novel galantamine carboxylate salts galantamine gluconate and galantamine lactate both have solubilities in water of approximately 400 mg/mL (1.39 M). Typical yields at the lab scale for the current ion exchange batch process are 89-97%. The examples listed below provide additional details of the methodology and the experimental data.

A batch process is a process in which the feed is charged into the system at the beginning of the process, and the products are removed all at once some time later. No mass crosses the system boundaries between the time the feed is charged and the time the product is removed.

In a continuous process inputs and outputs flow continuously throughout the duration of the process.

With the concentration barrier removed, formulation of galantamine for nasal delivery can now be achieved without the addition of excipients (e.g., solubility enhancers) to address solubility concerns. The small volume necessary for nasal delivery is no longer a limiting factor for galantamine intranasal formulation development. Even the highest dose generally delivered orally, 24 mg galantamine, can feasibly be delivered in a single nasal spray dose of 100 μL.

These salts of galantamine have a 10 fold increase in solubility compared to galantamine hydrobromide. These galantamine salts can be administered to an individual to inhibit acetylcholinesterase in the treatment of such diseases as Alzheimer's disease, atony of the smooth muscle of the intestinal tract and urinary bladder, glaucoma, myasthenia gravis, and termination of the effects of competitive neuromuscular blocking drugs. A suitable dosage is 16-32 mg given twice a day.

### EXAMPLE 1

### “Galantamine Salt Exchange: Bromide to Gluconate Using QAE SEPHADEX® Slurry in a Batch Ion-Exchange Process”

Galantamine gluconate was produced according to the following procedure.

Study Design:

| Sample | Composition | Comments | Testing |
|---|---|---|---|
| 1 (100 mg/4 mL) | 25 mg/mL Galantamine HBr | | pH |
| | | | HPLC |
| | | | Elemental |
| | | | Analysis |

Methods and Materials:

Materials:

| | | | F.W. |
|---|---|---|---|
| Reagent | Grade | Vendor | Data |
| Galantamine HBr | | Tocris Cookson | 377.28 |
| Purified Water | | | |
| QAE SEPHADEX ® | | Pharmacia | |
| Sodium Gluconate | USP | Spectrum | 218.14 |

QAE SEPHADEX® Preparation:

QAE SEPHADEX® has a meq/g of 3.0+/−0.4. To be sure that the anion exchange sites were in a 100 fold excess of galantamine, 8.88 g dry powder QAE Sephadex was pre-swollen in water for 2 days at room temperature in a 250 mL beaker. (See the following chart for calculations to determine amount of QAE SEPHADEX® required.)

| Galantamine HBr | | | QAE SEPHADEX ® | | | |
|---|---|---|---|---|---|---|
| total mg | MW | moles | fold excess | eq | eq/g | g |
| 100 | 377.28 | 0.000265 | 100 | 0.026506 | 0.003 | 8.835 |
| 50 | 377.28 | 0.000133 | 100 | 0.013253 | 0.003 | 4.418 |

After QAE SEPHADEX® was swollen, it was rinsed with 1M Sodium Gluconate, pH 5.0 three times to fully bind gluconate to all the anion binding sites. The slurry was then be washed three times with purified water to remove excess salt in solution.

Galantamine Sample Preparation:

A 25 mg/mL Galantamine HBr solution was prepared by adding 100 mg galantamine to 4 mL purified water. Solution was vortexed to dissolve galantamine.

Ion Exchange:

After QAE Sephadex resin had been prepared, the galantamine HBr solution was added to the batch resulting in bromide ions binding to the QAE SEPHADEX® in the slurry and gluconate eluting off the resin and becoming complexed with the galantamine. The solution was left in the slurry for 30 min, with mild agitation at room temperature. The galantamine gluconate was recovered from the resin by filtration. Samples were centrifuged to clear any particles from the resin that are in solution.

Removing Water:

Samples were lyophilized using the BenchTop 2K lyophilizer from Virtis (Gardner, N.Y.). Samples were dried in 50 mL centrifuge tubes to maximize surface area space.

Solubility Test:

Dried galantamine was weighed in 50 mL. A minimum volume of purified water was be added to each sample slowly to maximize concentration of galantamine in solution. After Galantamine was dissolved in water, the solution was removed from the 50 mL tube and the tube was weighed again to determine the amount of galantamine in the tube by weight loss. The final concentration was determined by HPLC.

HPLC Methods:

All samples (and corresponding “placebos”) were diluted 1:150 with 50 mM ammonium formate.

Samples were assayed using an isocratic LC (Waters Alliance) method with UV detection.

| | |
|---|---|
| Column: | Waters Symmetry Shield, C18, 5 um, |
| | 25 × 0.46 cm |
| Mobile phase: | 1.5% ACN in 50 mM ammonium formate, pH 3.0 |
| Flow rate: | 1.3 mL/min |
| Column temperature: | 30° C. |
| Calibration curve: | 0-400 μg/mL Galantamine HBr (Tocris) |
| Detection: | UV at 285 nm |

Results:

Using the above-described procedure resulted in a 98.23% recovery of galantamine gluconate. The solubility of the galantamine gluconate was at least 238 mg/mL, which was at least approximately a 5.75 fold increase in solubility over the solubility of galantamine hydrobromide. Elemental analysis confirmed a 263-fold reduction in the ratio of bromide to galantamine, confirming that the bromide salt was successfully exchanged.

### EXAMPLE 2

### “Galantamine Salt Exchange: Bromide to Lactate Using QAE SEPHADEX® Slurry in a Batch Ion-Exchange Process”

Study Design:

| Sample | Composition | Testing |
|---|---|---|
| 1 (100 mg/4 mL) | 25 mg/mL Galantamine HBr | pH |
| | | HPLC |
| | | Elemental |
| | | Analysis |

Methods and Materials:

Materials:

Materials:

| Reagent | Grade | Vendor | F.W. |
|---|---|---|---|
| Galantamine HBr | | Tocris Cookson | 377.28 |
| Purified Water | | | |
| QAE SEPHADEX ® | | Pharmacia | |
| Sodium Lactate Solution, 60% | USP | Spectrum | 112.06 |

Galantamine lactate was produced according to the same procedure that galantamine gluconate was produced except that sodium lactate was the carboxylate salt instead of sodium gluconate.

Results: The process described above produced an 89.74% yield of galantamine lactate. The solubility of the galantamine lactate was about 314 mg/mL, which was more than about a 9-fold increase in solubility over galantamine hydrobromide. Elemental analysis confirmed a 227-fold reduction in the ratio of bromide to galantamine, confirming that the bromide salt was successfully exchanged.

### EXAMPLE 3

### Exchanging Salt Forms of Galantamine-Gluconate for Bromide and Lactate for Bromide

Study Design:

| Sample | Composition | Comments | Testing |
|---|---|---|---|
| 1 (200 mg/8 mL) | 25 mg/mL Galantamine HBr | Gluconate | pH |
| | | salt | HPLC |
| | | exchange | EA |
| 1 (200 mg/8 mL) | 25 mg/mL Galantamine HBr | Lactate salt | pH |
| | | exchange | HPLC |
| | | | EA |

Methods and Materials:

Materials:

Materials:

| | | | F.W. |
|---|---|---|---|
| Reagent | Grade | Vendor | Data |
| Galantamine HBr | | Tocris Cookson | 377.28 |
| Purified Water | | | |
| QAE sephadex | | Pharmacia | |
| Sodium Gluconate | USP | Spectrum | 218.14 |
| Sodium Lactate Solution, 60% | USP | Spectrum | 112.06 |

QAE SEPHADEX® Preparation:

QAE SEPHADEX® has a meq/g of 3.0+/−0.4. To be sure that the anion exchange sites were in a 100 fold excess of galantamine, 2 separate aliquots of 17.6 g dry powder QAE SEPHADEX® were pre-swollen in water for 2 days at room temperature. (See the following chart for calculations to determine amount of QAE SEPHADEX® required.)

| Galantamine HBr | | | QAE sephadex | | | |
|---|---|---|---|---|---|---|
| total mg | MW | moles | fold excess | eq | eq/g | g |
| 100 | 377.28 | 0.000265 | 100 | 0.026506 | 0.003 | 8.835 |
| 200 | 377.28 | 0.000530 | 100 | 0.053011 | 0.003 | 17.67 |

After the QAE SEPHADEX® was swollen, it was rinsed with either 1M sodium gluconate three times or 1 M Sodium Lactate four times to fully bind gluconate or lactate to all the anion binding sites. The slurry was then washed three times with purified water to remove excess salt in solution.

Galantamine Sample Preparation:

Two 25 mg/mL Galantamine HBr solutions were prepared by adding 200 mg galantamine to 8 mL purified water. The solutions were vortexed to dissolve the galantamine.

Ion Exchange:

After the QAE SEPHADEX® resin was prepared, the Galantamine HBr solution was added in batch. Bromide ion bound to QAE SEPHADEX® and gluconate or lactate complexed with galantamine. The solution was left on the beads for 60 min, and mildly agitated at room temperature. The galantamine gluconate or galantamine lactate were recovered from the resin by filtration. Multiple fractions were collected from the resin by adding water to the resin after the initial sample was collected. This is to maximize galantamine recovery. Samples will be centrifuged to clear any particles from the resin that are in the recovered fractions. Concentration was determined by HPLC.

Removing Water:

Samples were lyophilized using the BenchTop 2K lyophilizer from Virtis (Gardner, N.Y. model # 393775). Samples were dried in 50 mL centrifuge tubes to maximize surface area space.

Solubility Test:

Dried galantamine in 50 mL tubes will be weighed. A minimum volume of purified water will be added to each sample slowly to maximize concentration of galantamine in solution. After Galantamine has dissolved in water, the solution will be removed from the 50 mL tube and the tube will be weighed again to determine the amount of galantamine in the tube by weight loss.

HPLC Methods:

All samples (and corresponding “placebos”) diluted 1:150 with 50 mM ammonium formate, pH 3.0, i.e. 10 μL sample mixed with 1490 μL diluent.

Samples assayed using an isocratic LC (Waters Alliance) method with UV detection.

| | |
|---|---|
| Column: | Waters Symmetry Shield, C18, 5 um, |
| | 25 × 0.46 cm |
| Mobile phase: | 1.5% ACN in 50 mM ammonium formate, pH 3.0 |
| Flow rate: | 1.3 ml/min |
| Column temperature: | 30° C. |
| Calibration curve: | 0-400 μg/mL Galantamine HBr (Tocris) |
| Detection: | UV at 285 nm |

Results: The process described above produced an 83% yield of galantamine lactate. The solubility of the galantamine lactate was at least 395 mg/mL, which was more than an 11-fold increase in solubility over galantamine hydrobromide. Furthermore, the process above-described process produced an 87% yield of galantamine gluconate. The solubility of the galantamine gluconate was at least 395 mg/mL, which was more than an 11-fold increase in solubility over galantamine hydrobromide.

### EXAMPLE 4

### Galantamine Salt Exchange: Bromide to Lactate Using a 1 mL Q SEPHAROSE® Column

| Sample | Composition | Comments | Testing |
|---|---|---|---|
| 1 (8 mg/266.7 μL) | 30 mg/mL | Lactate salt | UV(285 nm) |
| | Galantamine HBr | exchange | osm |
| | | 10 fold excess | conductivity |
| | | of resin | Br— ion |
| | | | HPLC |
| 2 (4 mg/133.3 μL) | 30 mg/mL | Lactate salt | UV(285 nm) |
| | Galantamine HBr | exchange | osm |
| | | 20 fold excess | conductivity |
| | | of resin | Br— ion |
| | | | HPLC |
| 3 (1.6 mg/53.3 μL) | 30 mg/mL | Lactate salt | UV(285 nm) |
| | Galantamine HBr | exchange | osm |
| | | 50 fold excess | conductivity |
| | | of resin | Br— ion |
| | | | HPLC |
| 4 (0.8 mg/26.7 μL) | 30 mg/mL | Lactate salt | UV(285nm) |
| | Galantamine HBr | exchange | osm |
| | | 100 fold excess | conductivity |
| | | of resin | Br— ion |
| | | | HPLC |

Methods and Materials:

Materials:

Materials:

| Reagent | Grade | Vendor | F.W. |
|---|---|---|---|
| Galantamine HBr | | Tocris Cookson | 377.28 |
| Purified Water | | | |
| HiTrap Q SEPHAROSE ® FF | | Amersham | |
| | | Biosciences | |
| Sodium Lactate Solution, 60% | USP | Spectrum | 112.06 |

HiTrap Q SEPHAROSE® FF Column Preparation:

HiTrap Q SEPHAROSE® FF columns were equilibrated following the instructions manual. First, a 1 mL column was washed with 5 column volumes of water to remove preservatives and storage buffer. The column was subsequently washed with 5 column volumes of 1 M sodium lactate to prime the column. Finally, the column was washed with 5-10 column volumes of water to remove the excess salt. Eluent was monitored with a conductivity meter to assess that all excess salt was no longer eluting from column.

Galantamine Sample Preparation:

1 mL of 30 mg/mL Galantamine HBr solution was prepared. The solutions were vortexed to dissolve galantamine.

To determine the minimal amount of excess resin that is required to successfully exchange galantamine HBr for galantamine lactate, varying amounts of galantamine were loaded on the 1 mL columns to test 50×, 20×, and 10× excess of the ionic capacity of Q SEPHAROSE® to the moles of galantamine present. The ionic capacity of the resin is 0.18-0.25 mmole/mL gel. (See the following chart for calculations to determine amount of galantamine to load on a 1 mL column.)

| Galantamine | | | | | |
|---|---|---|---|---|---|
| HBr | | excess resin | | Q SEPHAROSE ® | |
| mg | mmoles | fold | mmole | mL (low capacity) | mL (high capacity) |
| 1.6 | 0.004 | 50 | 0.212 | 1.18 | 0.85 |
| 8.0 | 0.021 | 10 | 0.212 | 1.18 | 0.85 |

Ion Exchange:

After the HiTrap Q SEPHAROSE® column was prepared, the Galantamine HBr solution was loaded with a syringe at approximately 1 mL/min. Bromide ion bound to Q SEPHAROSE® and lactate complexed with galantamine. The galantamine lactate was be eluted from the column by washing the column with 5-10 column volumes of water. Multiple 1 mL fractions were collected from the column to maximize galantamine recovery. Samples were tested for conductivity, osmolarity, pH, and for galantamine content by measuring A₂₈₅. Concentration was determined by HPLC.

Removing Water:

Samples were lyophilized using the BenchTop 2K lyophilizer from Virtis (Gardner, N.Y.). Samples (2-4 mL total vol) were dried in 15 mL centrifuge tubes to maximize surface area space.

Solubility Test:

A minimum volume of purified water was added to each sample slowly to maximize concentration of galantamine in solution. After Galantamine was dissolved in water, the solution was transferred to a microcentrifuge tube.

Measurement of Osmolarity:

Samples were measured with an Advanced Micro Osmometer, Model 3300, S/N 9812146H from Advanced Instruments Inc. (Norwood, Mass.) using a 20 microliter Sampler, and disposable sample tips.

Conductivity Measurements:

Conductivity was measured using the Traceable® Portable Conductivity Meter, with probe from VWR International.

Bromide Ion Concentration Determination:

Bromide ions were measured using an Ionplus Sure Flow Bromide probe, Orion model 9635BN with Orion 520Aplus pH meter, Thermo Electron Corp (USA).

UV Spectrophotometer Measurements:

UV absorbance were read on a μQuant optical density plate reader, by Biotek Instruments (Winooski, Vt.) at 285 nm using KCJr software. 100 μL of sample were loaded in each well. Water was used as a blank. To get an estimate of galantamine concentration, three controls were loaded: 0.333 mg/mL, 0.111 mg/mL and 0.055 mg/mL Galantamine HBr in water. From these, a line was plotted and the concentrations of the fractions from the columns were determined.

HPLC Methods:

Samples were assayed using a gradient LC (Waters Alliance) method with UV detection.

| | |
|---|---|
| Column: | Waters Symmetry Shield, C18, 5 um, 25 × 0.46 cm |
| Mobile phase: | A: 1.5% ACN in 50 mM ammonium formate, pH 3.0 |
| | B: ACN |
| Flow rate: | 1.3 mL/min |
| Column | 30° C. |
| temperature: | |
| Calibration curve: | 0-400 μg/mL Galantamine HBr (Tocris) |
| Detection: | UV at 285 nm |
| Sample diluent: | Buffer A |

Results: the process described above produced a 91% yield of galantamine lactate. The solubility of the galantamine lactate was at least 217 mg/mL, which was more than a 6-fold increase in solubility over galantamine hydrobromide. Detection of bromide ions using the bromide ion specific probe demonstrated about a 240-fold reduction in the ratio of bromide to galantamine, confirming that the bromide salt was successfully exchanged.

### EXAMPLE 5

### Galantamine Salt Exchange: Bromide to Gluconate Using a 1 ml Q SEPHAROSE® Column

Study Design:

| Sample | Composition | Comments | Testing |
|---|---|---|---|
| 2 (4 mg/133.3 μL) | 30 mg/mL | Galantamine | UV(285 nm) |
| | Galantamine HBr | HBr solution | conductivity |
| | | made for Gal-022 | Br- ion |
| | | Gluconate salt | HPLC |
| | | exchange 20 fold | |
| | | excess of resin | |

Methods and Materials:

Materials:

Materials:

| Reagent | Grade | Vendor | F.W. |
|---|---|---|---|
| Galantamine HBr | | Tocris Cookson | 377.28 |
| Purified Water | | | |
| HiTrap Q SEPHAROSE ® FF | | Amersham | |
| | | Biosciences | |
| Sodium Gluconate | USP | Spectrum | 218.14 |

Galantamine gluconate was produced according to the procedure of Example 4 except that sodium gluconate was the carboxylate salt instead of sodium lactate.

Results: The process described above produced a 99% yield of galantamine gluconate. The solubility of the galantamine gluconate was at least 215 mg/mL, which was more than a 6-fold increase in solubility over galantamine hydrobromide. Detection of bromide ions using the bromide ion specific probe demonstrated about a 228-fold reduction in the ratio of bromide to galantamine, confirming that the bromide salt was successfully exchanged.

### EXAMPLE 6

### Galantamine Salt Exchange: Bromide to Lactate on a 1 L Q SEPHAROSE® Column

Study Design:

STUDY DESIGN:

| Sample | Composition | Comments | Testing |
|---|---|---|---|
| 1 (1 g/33.3 mL) | 30 mg/mL | Lactate salt | UV(285 nm) |
| | Galantamine HBr | exchange 100 fold | osm |
| | | excess of resin | conductivity |
| | | | Br- ion |
| | | | HPLC |

Methods and Materials:

Materials:

Materials:

| Reagent | Grade | Vendor | F.W. |
|---|---|---|---|
| Galantamine HBr | | Tocris Cookson | 377.28 |
| Purified Water | | | |
| Q SEPHAROSE ® FF | | Amersham | |
| | | Biosciences | |
| Sodium Lactate Solution, 60% | USP | Spectrum | 112.06 |

SEPHAROSE® FF Column Packing:

A column was first packed in an XK50/60 column body from Amersham Biosciences with Q SEPHAROSE® Fast Flow resin, according the instructions from Amersham. Briefly, the 20% Ethanol solution was decanted from the Q SEPHAROSE® resin and a slurry was prepared that contains roughly 75% resin and 25% water. The resin was then be degassed under a vacuum. The Column was prepared by flushing the bottom with water to purge the system of air. The column was packed with the addition of a RK50 reservoir from Amersham. The degassed resin was poured in one smooth motion down the length of the column along a side wall. The column was attached to a BioRad Econo Pump peristaltic pump (s/n 700 BR 09961). The upper limit for a linear flow rate for the resin, as quoted in the instructions, is 400-700 cm/hr. The maximum flow rate for this pump is 20 mL/min.

Column Pre-Washes:

Once the column bed was packed and a constant bed height reached, the adaptor was attached and the column was washed with 3-5 column volumes of water. The eluant was monitored by conductivity to confirm that the column reached equilibrium.

After the first wash with water, the column was washed with 1 M Sodium Lactate for 5 column volumes or until the conductivity of the eluant ceased to change and matched that of the solution being loaded on the column. The 1 M sodium lactate was degassed before use. The flow rate was 12 mL/min.

After the 1 M salt wash, the column underwent a second water wash to remove excess salt from the column. The water was degassed before use. The eluant was monitored by conductivity and this step continued until either 10 column volumes of water were used or the conductivity dropped below 30 μS/cm. The flow rate was 12 mL/min.

Galantamine Sample Preparation:

33.3 mL of a 30 mg/mL Galantamine HBr solution was prepared. The solution was vortexed to dissolve the galantamine.

Ion Exchange:

After the Q SEPHAROSE® column was prepared, the Galantamine HBr solution was loaded. Bromide ion bound to the Q SEPHAROSE® and lactate complexed with the galantamine. The galantamine lactate was eluted from the column by washing the column with 2-5 column volumes of water. 7.5 mL fractions were collected from the column to maximize galantamine recovery and separation from excess salt. Samples were tested for conductivity, osmolarity, and for galantamine content by measuring A₂₈₅. Concentration were determined by HPLC.

Removing Water:

Samples were lyophilized using the BenchTop 2K lyophilizer from Virtis (Gardner, N.Y. model # 393775. Samples (15-20 mL total vol) were dried in 40 mL glass vials to maximize surface area space.

Solubility Test:

A minimum volume of purified water was added to each sample slowly to maximize concentration of galantamine in solution. After galantamine was dissolved in water, the solution was transferred to a microcentrifuge tube.

Measurement of Osmolarity:

Samples were measured with an Advanced Micro Osmometer, Model 3300, from Advanced Instruments Inc. (Norwood, Mass.) using a 20 microliter Sampler, and disposable sample tips.

Conductivity Measurements:

Conductivity was measured using the Traceable® Portable Conductivity Meter, with probe from VWR International.

Bromide Ion Concentration Determination:

Bromide ions were measured using an Ionplus Sure Flow Bromide probe, Orion model 9635BN with Orion 520Aplus pH meter, Thermo Electron Corp (USA).

UV Spectrophotometer Measurements:

UV absorbance was read on a μQuant optical density plate reader, by Biotek Instruments (Winooski, Vt.) at 285 nm using KCJr software. 100 μL of sample will loaded in each well. Water was used as a blank. To get an estimate of galantamine concentration, three controls were loaded: 0.333 mg/mL, 0.111 mg/mL and 0.0370 mg/mL Galantamine HBr in water. From these, a line was plotted and the concentrations of the fractions from the columns were determined.

HPLC Methods:

Samples were assayed using a gradient LC (Waters Alliance) method with UV detection.

| | |
|---|---|
| Column: | Waters Symmetry Shield, C18, 5 um, 25 × 0.46 cm |
| Mobile phase: | A: 1.5% ACN in 50 mM ammonium formate, pH 3.0 |
| | B: ACN |
| Flow rate: | 1.3 ml/min |
| Column | 30° C. |
| temperature: | |
| Calibration curve: | 0-400 μg/mL Galantamine HBr (Tocris) |
| Detection: | UV at 285 nm |
| Sample diluent: | Buffer A |

### RESULTS

Galantamine Recovery from Column:

Every 5^{th} fraction was monitored by conductivity and by UV absorbance at 285 nm. Results are shown in the graph on the next page:

766.8 mg Galantamine (1.000 mg Galantamine HBr) was loaded on the column.

| | | | | Total | | | |
|---|---|---|---|---|---|---|---|
| | | volume | Galantamine | Galantamine | | Bromide | Bromide |
| Pool | Fractions | (mL) | (mM) | (mg) | Recovery % | (mM) | remaining % |
| A | 30-44 | 109 | 0.5 | 15.0 | 2.0% | 0.0013 | 0.27% |
| B | 45-59 | 109 | 1.9 | 59.5 | 7.8% | 0.0026 | 0.14% |
| C | 60-69 | 71 | 7.1 | 144.9 | 18.9% | 0.0084 | 0.12% |
| D | 70-80 | 79 | 22.1 | 501.7 | 65.4% | 0.0244 | 0.11% |
| E | 83-90 | 57 | 1.4 | 22.9 | 3.0% | 0.0030 | 0.22% |
| | | | Total (mg): | 744.0 | 97.0% | | |

Galantamine concentration determined by HPLC

Bromide concentration determined by bromide ion probe

The teachings of all of the references, patents and patent applications cited herein are incorporated herein by reference.

What is claimed is:

1. A galantamine carboxylate salt.

2. The galantamine carboxylate salt of claim 1 having a carboxylate anion associated with a galantamine cation in solution.

3. The galantamine carboxylate salt of claim 2 wherein the carboxylate anion contains one or more hydroxyl groups.

4. A galantamine carboxylate salt selected from the group consisting of galantamine gluconate, galantamine lactate, galantamine citrate and galantamine glucarate, galantamine benzoate, galantamine acetate, galantamine salicylate, galantamine tartrate, galantamine mesylate, galantamine tosylate, galantamine maleate, galantamine fumarate, and galantamine stearate.

5. A method for producing a galantamine carboxylate salt comprising:
forming a solution of a carboxylate salt forming carboxylate anions in solution;
applying the solution to an anion exchange resin under conditions wherein the carboxylate anions bind to the anion exchange resin; forming a solution of galantamine hydrobromide under conditions where bromide ions are formed in solution; applying the galantamine hydrobromide solution to the anion exchange resin under conditions wherein the carboxylate anions are displaced and the bromide anions bind to the anion exchange resin resulting in the formation of a galantamine carboxylate salt or complex.

6. The method of claim 5 wherein the process is conducted in a batch process.

7. The method of claim 6 wherein the batch process is a slurry of anion exchange resin.

8. The method of claim 6 wherein the batch process uses an anion exchange resin packed into a column.

9. The method of claim 8 wherein the process is conducted as a continuous process using an anion exchange resin packed into a column.

10. The method of claim 5 wherein the carboxylate salt is selected from the group consisting of a gluconate salt, an acetate salt, a citrate salt and a glucarate salt.

11. The method of claim 10 wherein the process is conducted in a batch process.

12. The method of claim 11 wherein the batch process is a slurry of the anion exchange resin.

13. The method of claim 11 wherein the batch process uses an anion exchange resin packed into a column.

14. The method of claim 13 wherein the process is conducted as a continuous process using an anion exchange resin packed into a column.

15. A method for inhibiting acetylcholinesterase in a mammal comprising administering to said individual a galantamine carboxylate salt.

16. The method of claim 15 wherein the mammal is a human.

17. The method of claim 15 wherein the galantamine carboxylate salt is selected from the group consisting of galantamine gluconate, galantamine lactate, galantamine citrate and galantamine glucarate, galantamine benzoate, galantamine acetate, galantamine salicylate, galantamine tartrate, galantamine mesylate, galantamine tosylate, galantamine maleate, galantamine fumarate, and galantamine stearate.

18. The method of claim 17 wherein the mammal is a human.

19. The method claim 15 wherein galantamine carboxylate salt has carboxylate anion associated with a galantamine cation in solution.

20. The method of claim 19 wherein the mammal is a human.

21. The method of claim 19 wherein the carboxylate anion contains one or more hydroxyl groups.

22. The method of claim 21 wherein the mammal is a human.
